(19) United States
(12) Patent Application Publication
(10) Pub. No.: US 2002/0032422 A1
(43) Pub. Date: Mar. 14, 2002

(54) WASHABLE ABSORPTION ELEMENT SUITABLE FOR REUSE

(76) Inventor: GREGORIUS M.H. GOYARTS, OISTERWIJK (NL)

Correspondence Address: Troutman Sanders LLP, Bank of America Plaza, 600 Peachtree Stree NE, Suite 5200, Atlanta, GA, 30308-2216, US

(21) Appl. No.: 09/142,966

(22) PCT Filed: Mar. 19, 1997
(86) PCT No.: PCT/NL97/00141

(30) Foreign Application Priority Data
Mar. 20, 1996 (NL) 1002659

(51) Int. Cl. A61F 13/15; A61F 13/20; A61M 1/00

(52) U.S. Cl. 604/367; 604/385.15; 604/385.25; 604/385.29; 604/391

(57) ABSTRACT

A washable absorption element (1) is

[chemical structure]

described which is suitable for reuse. This absorption element comprises at least one liquidabsorbing layer (2) which comprises a mixture of manmade and synthetic fibers. At its upper side and underside, respectively, the liquidabsorbing layer is bonded to one or more liquidtransporting layers (3,4) of nonwoven material which substantially comprises synthetic fibers. A method for fabricating the above absorption element is likewise described. The use of the absorption element and articles comprising such an absorption element is like wise described. Finally, a nappy is described which comprises a washable outer layer of liquidimpermeable material and the abovementioned absorption element.

The present invention relates to a washable absorption element suitable for reuse, at least comprising a liquids-absorbing layer which comprises a mixture of man-made and synthetic fibres.

Such an absorption element is disclosed by the international application WO 93/18728. This publication describes a washable nappy which is suitable for reuse and which comprises a disposable layer for (solid) faeces. The nappy additionally comprises an absorbent body which consists of man-made and synthetic fibres, viz. viscose and polyester, and which is placed between the outer layer of the nappy and a covering layer on the inside of the nappy. The absorbent body is bonded to the covering layer by means of stitched seam.

The German Gebrauchsmuster G 9201050.4 likewise discloses a nappy comprising an absorption layer in which man-made and synthetic fibres, viscose and polyester, are present. In this case, however, the viscose/polyester layers are each surrounded by a cotton fabric. A drawback of the use of cotton in an absorption body is that cotton has a fairly low absorption capacity and spreading capacity. If the absorption element is used in, for example, a nappy, this will mean that liquids (i-e. urine) continuously remain in contact with the skin, which is undesirable.

In the nappy according to WO 93/18728 the use of cotton in the absorption element is avoided in order thus to effect better absorption of liquids, compared with nappies into which cotton is incorporated. It was found, however, that the spreading and rewetting capacity and also the absorption rate and absorption capacity of the absorption element according to WC 93/18728 are still insufficient for them to be able to compete with the popular disposable nappies. The latter have very good characteristics in terms of absorption capacity, absorption rate, spreading capacity and rewetting capacity, but they are very harmful to the environment and contribute significantly to the total amount of waste.

It is an object of the present invention to provide a washable absorption element in accordance with the preamble, which can be used for many purposes and which can be reused, said absorption element having a very good spreading and rewetting capacity as well as a very high absorption rate and absorption capacity.

To this end, the washable absorption element according to the present invention is characterized in that the liquid-absorbing layer of the absorption element comprises <100% such as 90-10% of man-made fibres and up to 100% such as 10-90% of synthetic fibres and at the upper side and underside, respectively, thereof is bonded to one or more liquid-transporting layers of non-woven material which mainly comprises synthetic fibres.

Expediently the portion of man-made fibres is 80-20% and the portion of synthetic fibres 20-80%. The mixture-character of the absorption element is such that, for certain applications, a minimal proportion of synthetic fibres, such as 0,01-1 wt %, is sufficient whereby the major proportion is man-made fibres.

Alteratively also a structure can be chosen comprising nearly 100% synthetic fibres and a very small proportion of man-made fibres. The percentages mentioned hereinbefore are on basis of weight.

The abovementioned liquid-transporting layers act not only as a transport layer but also as a liquid-spreading layer and a rewetting layer. In the absence of the one or more liquid-transporting layers liquid comes directly into contact with the liquid-absorbing layer. In general, the liquid will come into contact with the absorption layer at substantially one particular point. It is then important that the absorption layer should spread the liquid as much as possible, so that maximum absorption of the liquid over the entire layer will take place and no “leakage” will occur. The latter proves virtually impossible, unless the absorbent layer is made to be very thick. The present invention, however, provides a solution for this problem by providing the liquid-absorbing layer on both sides with one or more liquid-transporting layers. Owing to the spreading function of the liquid-transporting layer(s), liquid is distributed optimally over the entire surface of the liquid-absorbing layer, so that the liquid will not come into contact with the absorption layer at substantially one point, but instead the absorption layer will be utilized to the greatest possible extent. Owing to the very good spreading capacity of the absorption element according to the invention, not only the absorption rate but also the absorption capacity and the rewetting capacity of the absorption element are increased. owing to both the upper side and the underside of the liquid-absorbing layer being bonded to one or more liquid-transporting layers, both liquid from the upper side and liquid which has reached the underside of the absorption element are passed into the liquid-absorbing layer via the liquid-transporting layer. In the simplest embodiment of the absorption element, the liquid-absorbing layer is surrounded on both sides by a single liquid-transporting layer, It is also possible, however, for the liquid-transporting layer to comprise a plurality of separate layers which may differ from one another with respect to fibre composition and fibre dimensions.

In particular, the synthetic fibre is selected from the group consisting of polyester, polyamide, polyacryl, poly-ethylene and polypropene or modified form thereof, and most preferably the synthetic fibre comprises polyester. Modified forms of the synthetic fibre comprise, for example, bi-component fibres.

Preferably, the man-made fibre comprises viscose which may or may not be modified.

The use of synthetic and man-made fibres in an absorption element is disclosed, for example, by WO 93/18728. An absorption element having a liquid-absorbing layer of man-made and synthetic fibres, which is surrounded on both sides by one or more liquid-transporting layers of non-woven material which mainly comprises synthetic fibres such as polyester fibres, has not been known until now.

So that the transport of liquid and the spreading thereof will proceed optimally, the liquid-transporting layers comprise polyester fibres having a thickness of 1-10 dTex and a length of 30-100 mm.

In a particular embodiment the liquid-transporting layers additionally comprise polyester having a thickness of less than 1 dTex and a length of 30-80 mm.

So as to effect optimum absorption capacity of the absorption element, the viscose in the liquid-absorbing layer preferably comprises fibres having a thickness of 1-8 dTex and a length of 30-100 mm, and the polyester in the liquid-absorbing layer comprises fibres having a thickness of 1-10 dTex and a length of 30-100 mm.

In a special embodiment, the polyester in the liquid-absorbing layer additionally comprises polyester having a thickness of less than 1 dTex and a length of 30-80 mm.

In particular, the liquid-absorbing layer of the absorption element comprises 60-90% of viscose and 40-10% of polyester and preferably 75-85of viscose and 25-15% of polyester.

In a preferred embodiment, the liquid-transporting layer and the liquid-absorbing layer of the absorption element are bonded to one another by means of interpenetration of the fibres from the one into the other layer and vice versa. Preferably, the liquid-transporting layer and the liquid-absorbing layer are bonded to one another by means of needling. As a result of the respective layers thus being bonded to one another, the optimum transport, absorption and spreading characteristics of the respective layers are retained. Of course it is also possible for the respective layers to be bonded to one another with other means. A more detailed description of such a bonding method is given hereinafter.

It will be understood that the various layers of the absorption element may have different thicknesses, depending on the application. Since in the case of the absorption element according to the present invention, the liquid-absorbing layer is used to its maximum potential, this layer does not have to be made unnecessarily thick in order to prevent leakage. In general, the liquid-absorbing layer preferably has a thickness of between 0.5 and 25 mm, while the liquid-transporting layers have a thickness of between 0.25 and 10 mm.

The present invention also relates to a method for fabricating an absorption element according to the present invention. Such a method comprises the following steps:

providing at least one liquid-absorbing layer and at least two liquid-transporting layers;

bonding, by means of needling, a liquid-transporting layer to the one side of the liquid-absorbing layer,

bonding, by means of needling, the other liquid-transporting layer to the opposite side of the liquid-absorbing layer and

optionally imparting the desired size to the absorption element.

In particular, the respective layers are subjected to a pretreatment before they are bonded to each other.

Preferably, such a pretreatment comprises needling of the respective layers.

In the course of such a pretreatment one of the layers may optionally be treated more intensively than the others.

So that shrinking of the absorption element is prevented as far as possible, the absorption element for is advantageously subjected to a thermofixation treatment, at maximally 185° C. In particular the absorption element is strengthened with a polyester-scrim or -gelege in order to optimize the stability and to reduce the shrinkage.

Such a treatment reduces shrinkage of the absorption element from 5-15% to 1-4%. A hitherto customary method for overcoming such a problem employs quilting.

Quilting involves different layers being bonded to one another with the aid of stitching patterns or stitching points. The drawback of quilting, however, is that the absorption capacity is greatly reduced at the location of the stitched seams.

The present invention also relates to an above-described absorption element which at the upper side and the underside is surrounded by an over- and underlay, respectively, of liquid-permeable material.

The over- and underlays comprise generally synthetic fibres such as polyester, polypropylene, polyamide, polyethylene, polyacryl or modified forms thereof. The structure of the over- and underlays may be of any form such as a knit, fabric, non-woven and the like. A polyester knit is preferred whereby expediently the knit has been raised and subsequently sheared.

Preferably, the overlay and the underlay comprise polyester. Furthermore, the layers, in an advantageous embodiment, may comprise not only the standard polyester but also polyester having a thickness of less that 1 dTex. Thus and by specific finishing of at least the overlay it is possible to ensure that this layer has a softer and more pliable handle, does not remain moist and exhibits breathable characteristics. Preferably, particularly for the overlay, material is used which has bactericidal properties.

The present invention also relates to the use of the absorption element according to the invention in an article, to impart absorbent characteristics to this article.

The invention further relates to an article comprising an absorption element according to the invention as set forth hereinabove.

The absorption element according to the present invention has a large number of applications, the applications mentioned hereinafter and the articles obtained therefrom being particularly preferred. These are the use in or for the packaging industry, furniture industry, automotive industry, agricultural and horticultural bonded fibre webs, bonded fibre webs for roofing, insulation materials, drainage media, filter materials, fume cupboards, fire blankets, absorption blankets, clothing such as sports clothing and sports accessories, also workwear, protective material against, for example, oil (in this case a laminated film can be applied to the absorption element), horse blankets, saddlecloths, absorption material for animal cages and the like, such as, for example, cat litter, cleaning articles, incontinence and decubitus materials, mattress covers, bandaging materials, and blankets to play on and to be used in playpens, for children.

The invention also relates to a nappy which at least comprises a washable outer layer of liquid-impermeable material and a washable absorption element according to the present invention, which preferably, on the upper side and the underside, is surrounded by an over- and underlay, respectively, of liquid-permeable material, the washable outer layer in the unused state being substantially elongate and being of such a shape that, when it is used, a substantially pant-shaped nappy is formed which is provided with two leg openings. The liquid permeable layers have preferably been provided with antibacterially active components. Such a nappy may be intended for babies or alternatively for sufferers from incontinence.

The absorption element according to the invention may in some way, detachable or not, be joined to the nappy pant or may be loosely inserted therein. In the latter case the nappy pant will normally comprise elements underneath which or against which the absorption element can be pushed, so that no shifting of the absorption element will take place while the nappy is in use.

In a particular embodiment the washable outer layer of the nappy comprises a polyester substrate which on at least one side is coated with a liquid-impermeable polyurethane layer which may or may not be air- and vapour-permeable.

This composition of the outer layer of the nappy ensures, on the one hand, the desired impermeability to liquid of the outer layer, while on the other hand the material has a pleasant feel to it and is permeable to air and vapour: i.e. the material is “breathable”.

The polyester substrate mentioned hereinbefore and which forms part of the washable outer layer is expediently a knitted, raised and subsequently sheared polyester substrate as has been described hereinbefore regards the upper- and underlays of the absorption element.

Advantageously, at the edge of the long side of the outer layer of the nappy an elastic sealing element is situated which, as seen in the direction of the width, has a side facing towards the outer layer and a side facing away from the cuter layer.

That side of the sealing element which faces towards the outer layer means that side of the sealing element which is closest to the outer layer, whereas the side facing away from the outer layer means the side which is furthest away from the outer layer.

Advantageously, the sealing element comprises a synthetic tape which is provided with at least one elastic thread which is positioned parallel to the longitudinal direction of the tape. Alternatively, however, other textile constructions can be used as a sealing element.

Preferably, the synthetic tape is provided with at least two elastic threads, at least that elastic thread which is substantially situated at the side facing away from the outer layer having a higher tension than the other elastic threads, Preferably such an elastic thread has been finished with a voluminous textured thread.

Such an increased tension ensures, both if one and if more elastic threads are used, that the outer edge of the elastic sealing element is moved upwards, so that the outer edge lies against the body of the person wearing the nappy.

To prevent moisture and faeces from leaking through the fabric of the elastic sealing element, the synthetic tape on at least one side is preferably provided at least in part with a liquid-impermeable layer of plastic, in particular a polyurethane layer. The liquid impermeable layers have been provided preferably with antibacterially active components. If such a polyurethane layer is provided for that side of the sealing element which faces away from the outer layer, said sealing element being brought into contact with the body by the tension of the elastic thread, the abovementioned leakage can be prevented. Employing the elastic thread having a higher tension together with the polyurethane or poly(vinyl chloride) results in a so-called “anti-leakage duct”.

In an advantageous embodiment, the tape of the sealing element is provided with at least three elastic threads positioned parallel to one another in the longitudinal direction, at least two of the elastic threads which are situated at the side facing towards the outer layer having threads situated between them, which are added in the longitudinal direction of the tape. Such added threads running in the longitudinal direction of the tape are also referred to as ends.

The presence, between the elastic threads near the outer layer, of a number of preferably closely spaced ends results in localized bulging of the tape. The skin of the person to which the nappy is applied, for example a baby, can work itself into that bulge and thus form a barrier against the leakage of moisture. Moreover, the use of such ends in combination with the higher tension of the outermost elastic thread ensures that the sides of the sealing element are bent towards the skin. An optimal seal of the nappy against the skin is thus ensured.

Advantageously, the synthetic tape comprises a polyester yarn.

Finally, the invention relates to an elastic sealing element as is described in the above.

The present invention will be explained in more detail with reference to the accompanying drawing, in which:

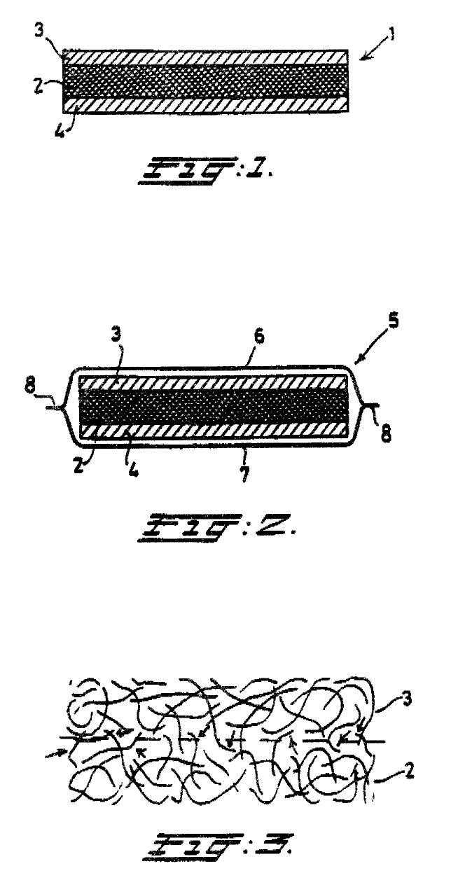

FIG. 1 schematically shows a section of an absorption element according to the invention;

FIG. 2 schematically shows a section of an absorption element which is surrounded by an over- and an underlay;

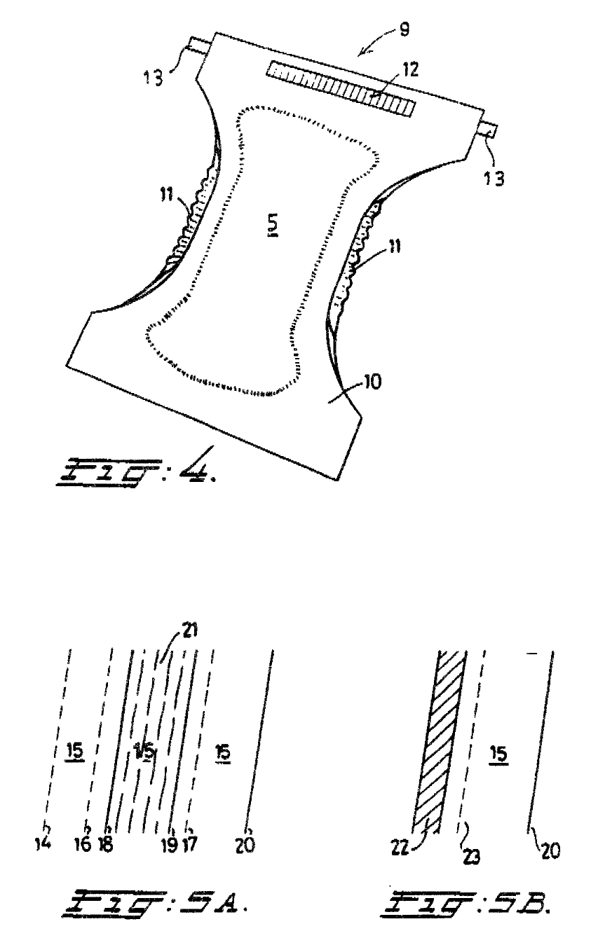

FIG. 3 schematically shows how the respective layers of the absorption element are bonded to one another;

FIG. 4 schematically shows a nappy according to the invention and used therein the absorption element according to the invention; and

FIGS. 5A, B schematically show an elastic sealing element according to the present invention.

FIG. 1 shows a section of an absorption element 1 which, in a so-called sandwich structure, comprises a liquid-absorbing layer 2 and, on both sides thereof, liquid-transporting layers 3 and 4. In the present case, the liquid-absorbing layer comprises 50-80% of viscose and 20-50% of polyester, 0.01-40% of the polyester optionally being polyester having a thickness of less than 1 dTex. The standard polyester fibres have an average thickness of 7 dTex and an average length of 60 mm, whereas the viscose fibres have an average thickness of 3 dTex and a length of 60 mm. The liquid-transporting over- and underlay 3, 4 mainly comprise polyester, a portion thereof, 0.01-40%, optionally being polyester having a thickness of less than 1 dTex. The polyester fibres have an average thickness of 7 dTex and a length of 60 mm. As previously observed, it is also possible for the liquid-transporting over- and underlay 3, 4 each to comprise a plurality of layers which may differ from one another in terms of fibre thickness and fibre length. It is possible to ensure, by selecting the correct ratios, that optimum transport of liquid will take place via the liquid-transporting layers.

FIG. 2 shows an absorption element 5, comprising the absorption element according to FIG. 1, which is surrounded by an over- and underlay of liquid-permeable material, indicated respectively by 6 and 7. This over- and underlay 6, 7 in the present case are made of polyester.

These layers 6, 7 are prefastened, with the aid of an either temporary or permanent bond, to the upper side 3 and underside 4, respectively, of the absorption element, and the polyester over- and underlay are then bonded to one another all around along edge 8. The bonding of the over- and underlay 6, 7 may take place by means of stitching or by means of thermal or chemical bonding, such as, for example, ultrasonic or high frequency welding or sealing.

It is also possible for the over- and underlay 6. 7 to be bonded to one another together with the absorption element 1.

FIG. 3 shows the manner in which the respective layers of the absorption element are bonded to one another. FIG. 3 depicts on an enlarged scale only the layers 3 and 2 of the absorption element. Arrows indicate at what points the fibres of the one layer have penetrated into the other layer and vice versa, so that bonding of the respective layers is achieved. This mutual interpenetration of the fibres is achieved by needling, needles being inserted into the layers and being withdrawn, so that the fibres are partially moved from the one into the other layer and vice versa.

FIG. 4 schematically shows a nappy 9 according to the present invention. The outer layer 10 of this nappy is substantially elongate, in the unused state, and when used, for example as a baby nappy, is pant-shaped. Reference number 11 shows elastic sealing elements which are present along the edges of the outer layer 10 of the nappy on the long side. These elastic sealing elements 11 ensure a good seal of the nappy against the body of, for example, a baby. The schematic structure of the elastic sealing element is explained in more detail in FIGS. 5A and 5B. On the inside of the outer layer 10 of nappy 9 there is the absorption element 5 according to the present invention. This absorption element 5 may be joined, detachably or non-detachably, to the outer layer 10. It is also possible for the absorption element 5 to be inserted loosely into the outer layer 10. To this end the nappy may, for example, be provided with overhanging edge sections along the circumference, within which the absorption element can be accommodated. Thus the absorption element 5 can be replaced after same time and be washed, while the outer layer 10 remains in use over a longer period. In the embodiment, shown in FIG. 4, of the outer layer 10 of the nappy 9 the latter is provided with a closure system in the form of velcro tape 13 for closing the nappy. The outer layer may also be provided with a rear elastic 12 so as to ensure a complete seal of the nappy against the skin of the baby. The velcro tape parts are preferably designed such that they also can close against themselves in order to prevent them from adhering against other laundry during washing (washing-safety system).

FIGS. 5A and 5B schematically depict the structure of two embodiments of the elastic sealing element 11 according to the invention, which is located on the long side of the outer layer of the nappy. Reference number 14 in FIG. 5A shows the non-elastic edge of the synthetic tape 15. This edge defines that side of the sealing element 11 which faces towards the outer layer 10. The synthetic tape 15 in FIG. 5A extends up to the elastic edge 20 of the tape. This elastic edge 20 defines that side of the sealing element 11 which faces away from the outer layer 10. In the present case the synthetic tape 15 comprises a polyester/polyamide tape into which some elastomer has been incorporated. Reference numbers 16 and 17 denote garment-welding manufacturing marks. In this case, three elastic threads are present in the synthetic tape, which are indicated, respectively, by 18, 19 and 20. If the sealing element according to FIG. 5 is applied to the outer layer of a nappy or is formed integrally therewith, the side indicated by reference number 14 (edge of the tape) will be situated near the side of the nappy. Between the elastic threads 18 and 19, ends 21 are positioned which are located in the longitudinal direction of the tape, a hollow structure thus being formed between the elastic threads 18 and 19. This structure makes it possible, when used, for example, as a baby's nappy, for the skin of the baby to work itself into the cavity so that a so-called water barrier is formed. In the present case, the synthetic tape 15 is coated on at least one side, between the elastic thread 19 and the elastic thread 20, with a polyurethane coating.

FIG. 5B depicts another embodiment of the sealing element 11. In this case, the synthetic tape 15 is joined, at the side facing towards the outer layer 10, to an elastic tape 22 which lies against the skin of, for example, the baby. Reference number 23 shows a garment-welding manufacturing mark. On the side facing away from the outer layer 10, the tape 15 as in the case of FIG. 5A is delimited by elastic thread 20. As a result of the tension of elastic thread 20 being made relatively high, the outer edge of the sealing element turns over and comes to lie against the skin of the baby. The edge formed between the elastic tape 22 and the elastic thread 20 can be coated with polyurethane to effect a barrier against moisture.

1. Washable absorption element (1), suitable for reuse, at least comprising a liquid-absorbing layer (2) which comprises a mixture of man-made and synthetic fibres, characterized in that the liquid-absorbing layer (2) comprises <100% such as 90-10% of man-made fibres and up to 100% such as 10-90% of synthetic fibres and at the upper side and underside, respectively, thereof is bonded to one or more liquid-transporting layers (3, 4) of non-woven material which substantially comprises synthetic fibres.

2. Absorption element according to claim 1, characterized in that the liquid-absorbing layer (2) thereof comprises 90-60% of man-made fibres and 10-40% of synthetic fibres.

3. Absorption element according to claim 1 or 2, characterized in that the synthetic fibre is selected from the group consisting of polyester, polyamide, polyacrylate and polypropene or modified forms thereof.

4. Absorption element according to one or more of claims 1-3, characterized in that the synthetic fibre comprises polyester.

5. Absorption element according to one or more of claims 1-3, characterized in that the man-made fibre comprises viscose which may or may not be modified.

6. Absorption element according to claim 4, characterized in that the liquid-transporting layers (3, 4). comprise polyester fibres having a thickness of 2-10 dTex and a length of 30-100 μm.

7. Absorption element according to claim 6, characterized in that the liquid-transporting layers (3, 4) additionally comprise polyester having a thickness of less than 1 dTex and a length of 30-80 mm.

8. Absorption element according to one or more of claims 4-7, characterized in that the liquid-absorbing layer (2) comprises viscose fibres having a thickness of 2.5-6 dTex and a length of 30-100 mm and comprises polyester fibres having a thickness of 2-10 dTex and a length of 30-100 mm.

9. Absorption element according to claim 8, characterized in that the polyester additionally comprises polyester having a thickness of less than 1 dTex and a length of 30-80 mm.

10. Absorption element according to one or more of claims 1-9, characterized in that the liquid-transporting layers (3, 4) and the liquid-absorbing layer (2) are bonded to one another by means of interpenetration of the fibres from the one into the other layer and vice versa.

11. Absorption element according to claim 10, characterized in that the liquid-transporting layers (3, 4) and the liquid-absorbing layer (2) are bonded to one another by means of needling.

12. Absorption element according to one or more of claims 1-11, characterized in that the liquid-absorbing layer (2) has a thickness of between 0.5 and 25 mm and the liquid-transporting layers (3, 4) have a thickness of between 0.25 and 10 mm.

13. Method for fabricating an absorption element according to one or more of claims 1-12, comprising the following steps.
providing at least one liquid-absorbing layer (2) and
at least two liquid-transporting layers (3, 4);
bonding, by means of needling, a liquid-transporting layer (3, 4) to the one side of the liquid-absorbing layer (2);
bonding, by means of needling, the other liquid-transporting layer (3, 4) to the opposite side of the liquid-absorbing layer (2); and
optionally imparting the desired size to the absorption element.

14. Method according to claim 13, characterized in that the respective layers are subjected to a pretreatment before they are bonded to each other.

15. Method according to claim 14,. characterized in that the pretreatment comprises needling.

16. Method according to one or more of claims 13-15, characterized in that the absorption element (1) formed is subjected to a thermofixation treatment.

17. Method according to claim 16, characterized in that the temperature during the thermofixation treatment is raised to max. 185° C.

18. Absorption element according to one or more of claims 1-12, which at the upper side and the underside is surrounded by an over- and underlay (6, 7), respectively, of liquid-permeable material.

19. Absorption element (5) according to claim 18, characterized in that the overlay and the underlay (6, 7) comprise polyester.

20. Use of the absorption element according to one or more of claims 1-12, 18 or 19 in an article to impart absorbent characteristics to this article.

21. Article comprising an absorption element according to one or more of claims 1-12, 18 or 19.

22. Nappy (9), at least comprising a washable outer layer (10) of liquid-impermeable material and an absorption element (5) according to claim 18 or 19, the washable outer layer in the unused state being substantially elongate and being of such a shape that, when it is used, a substantially pant-shaped nappy is formed which is provided with two leg openings.

23. Nappy according to claim 22, characterized in that the washable outer layer (10) comprises a polyester substrate which on at least one side is coated with a liquid-permeable polyurethane layer which may or may not be air- and vapour-permeable.

24. Nappy according to claim 22 or 23, characterized in that at the edge of the long side of the outer layer an elastic sealing element (11) is situated which, as seen in the direction of the width, has a side facing towards the outer layer and a side facing away from the outer layer.

25. Nappy according to claim 24, characterized in that the sealing element (11) comprises a synthetic tape which is provided with at least one elastic thread (18, 19, 20) which is positioned parallel to the longitudinal direction of the tape.

26. Nappy according to claim 25 characterized in that the synthetic tape is provided with at least two elastic threads (18, 19, 20 ), at least that elastic thread which is substantially situated at the side facing away from the outer layer having a higher tension than the other elastic threads .

27. Nappy according to claim 25 or 26, characterized in that the synthetic tape on at least one side is provided at least in part with a liquid-impermeable layer of plastic.

28. Nappy according to claim 27, characterized in that the liquid-impermeable layer of plastic comprises polyurethane.

29. Nappy according to one or more of claims 25-28, characterized in that the synthetic tape is provided with at least three elastic threads (18, 19, 20) positioned parallel to one another in the longitudinal direction, at least two of the elastic threads (18, 19) which are situated at the side facing towards the outer layer having threads (21) situated between them, which are added in the longitudinal direction of the tape.

30. Nappy according to claims 25-29, characterized in that the synthetic tape comprises a polyester tape.

31. Elastic sealing element (11) according to one or more of claim 23-29.
